(19) United States
(12) Patent Application Publication
(10) Pub. No.: US 2021/0353701 A1
(43) Pub. Date: Nov. 18, 2021

(54) ULTRAVIOLET LIGHT-INDUCED INFLAMMATION SUPPRESSING AGENT COMPRISING ALTERNATIVE AUTOPHAGY INDUCING AGENT

(71) Applicant: Shiseido Company, Ltd., Chuo-ku, Tokyo (JP)

(72) Inventors: Tatsuya HASEGAWA, Tokyo (JP); Masaya NAKASHIMA, Tokyo (JP); Masashi MIYAI, Tokyo (JP); Shigeomi SHIMIZU, Tokyo (JP); Shinya HONDA, Tokyo (JP)

(73) Assignee: Shiseido Company, Ltd., Chuo-ku, Tokyo (JP)

(21) Appl. No.: 17/290,664

(22) PCT Filed: Nov. 1, 2019
(86) PCT No.: PCT/JP2019/043163

§ 371 Date: Jun. 21, 2021

(30) Foreign Application Priority Data
Nov. 2, 2018 (JP) 2018-207589

(51) Int. Cl. A61K 36/53; A61P 29/00; A61K 36/899; A61K 36/65; A61K 36/82; A61K 36/738; A61K 36/38; A61K 36/185; G01N 33/50

(52) U.S. Cl. CPC A61K 36/53; A61P 29/00; A61K 36/899; A61K 36/65; G01N 33/502; A61K 36/738; A61K 36/38; A61K 36/185; A61K 36/82

(57) ABSTRACT

The present invention addresses the problem of providing an ultraviolet light-induced inflammation suppressing agent. The present invention is based on the finding that an alternative autophagy (Atg5/Atg7-independent autophagy) participates in the suppression of ultraviolet light-induced inflammation. Thus, the above problem is solved by providing an alternative autophagy inducing agent.

### TECHNICAL FIELD

The present invention relates to a suppressing agent of ultraviolet light-induced inflammation comprising an alternative autophagy-inducing agent as an active ingredient. The present invention further relates to an alternative autophagy inducing agent, to a screening method for an ultraviolet light-induced inflammation suppressing agent using alternative autophagy activity as an indicator, and to a method for evaluating resistance to ultraviolet light-induced inflammation in human skin.

### BACKGROUND

The mechanisms of decomposition and regeneration of cytoplasm components (such as organelles and cytoplasmic proteins) in cells include the ubiquitin-proteasome system which is responsible for selective proteolysis, and autophagy which is a general non-selective bulk decomposition system. Autophagy is also referred to as “autophagocytosis”, in which cytoplasm components to be subjected to decomposition are taken up by a bilayer membrane (isolation membrane), and the isolation membrane is then closed and fused with a lysosome, allowing the cytoplasm component contents to be decomposed. It has been shown that autophagy contributes not only to normal cellular metabolism but also to decomposition of proteins that are excessively produced in the cytoplasm due to exposure to certain types of stress, and also abnormal proteins, and it thus performs several physiological functional roles. Examples of diseases reported to be associated with autophagy include cancer, neurological disorders (such as amyotrophic lateral sclerosis, Alzheimer's disease and Parkinson's disease), hepatitis (acute hepatitis and chronic hepatitis), hepatic cirrhosis, infectious disease and immune disorders. Thus, development is ongoing for a medicament expecting for therapeutic effects against such diseases by moderating autophagy function, such as anticancer agents, antidementia drugs and neurodegenerative disease drugs

As research has progressed on the molecular mechanisms of autophagy, over 30 molecules associated with autophagy have been identified, among which Atg5, Atg7 and LC3 have come to be considered essential for autophagy to take place. It is thought that LC3 undergoes processing by Atg7 after its synthesis in the cytoplasm, participating in isolation membrane binding via a complex formed with Atg5. Recently reported research (NPL 1), however, indicates the existence of a type of autophagy that does not require these molecules, such autophagy being referred to as “alternative autophagy” or “Atg5/Atg7-independent autophagy” to distinguish it from the conventional type of Atg5/Atg7-dependent autophagy (also referred to simply as “Atg5-dependent autophagy”). Alternative autophagy is controlled by Rab9 that is involved only in alternative autophagy, in addition to Ulk1 and Beclin1 that are also involved in the conventional type of Atg5/Atg7-dependent autophagy. Since alternative autophagy is induced by cellular stress, failure in its mechanism is thought to lead to cancer or other conditions, and therefore anticancer agents utilizing alternative autophagy have been developed (PTL 1). According to research that is conducted with mice having knockout of Atg5 that is associated with the Atg5-dependent autophagy pathway, it was demonstrated that the Atg5-dependent autophagy pathway improves atherosclerosis which is one of the manifestations of inflammatory disease. On the other hand, it has also been reported that atherosclerosis onset is unchanged with heterozygotic loss of Beclin1, but that inflammation is accelerated with Atg5 knockouts (NPL 2). It has also been reported that the conventional type of autophagy inhibits inflammation in keratinocytes (NPL 4). NPL 4 shows that inducing inflammation with MALP-2 in Atg5-knockdown keratinocytes resulted in a drastic increase in the inflammatory cytokines TNF-α and IL-6 compared to the control (Atg5-non-knockdown). Thus, while the relationship between Atg5-dependent autophagy, which is a conventional type of autophagy, and inflammation has been partially elucidated, the relationship between alternative autophagy and inflammation is still not understood.

Ultraviolet rays are electromagnetic waves with a wavelength in the ultraviolet region, and are classified as long wavelength ultraviolet rays (UV-A) which are longer than about 320 nm, medium wavelength range ultraviolet rays (UV-B) of about 320 to about 280 nm, or short wavelength ultraviolet rays (UV-C) which are shorter than about 280 nm. UV-C rays are absorbed by the ozone layer and are therefore generally not present in sunlight reaching the ground. Thus, ultraviolet rays that arrive on the ground are generally composed of about 95% UV-A and about 5% UV-B. Ultraviolet rays are known to cause a number of adverse effects on the body including melanin pigment production, DNA damage, denaturation of elastic fibers, such as collagen or elastin, in the dermis layer, and generation of active oxygen, and from a beauty perspective they are well known to cause various adverse effects resulting in blemishes, wrinkles, sagging, skin browning and skin aging. Ultraviolet ray damage can generally be classified as acute damage or chronic damage. Acute damage includes sunburn, suntan, ultraviolet ray keratitis and lowered immunological function, and chronic damage includes wrinkles, blemishes, skin cancer and cataracts. Inflammation produced by ultraviolet rays is one cause of such acute and chronic damage, and suppressing the inflammation caused by ultraviolet rays is important for preventing and treating ultraviolet damage. Further elucidation of the mechanism of inflammation caused by ultraviolet rays is desired and there is a need for a method of finding substances that suppress inflammation according to the elucidated mechanism of inflammation caused by ultraviolet rays.

As mentioned above, alternative autophagy is a recently newly discovered autophagy pathway, and while research is being conducted on its effects on physiological mechanisms and on its related diseases, the amount of knowledge obtained thus far is still not sufficient. The relationship between ultraviolet ray causation and inflammation, in particular, is still completely unknown.

### CITATION LIST

### Patent Literature

[PTL 1] International Patent Publication No. 2013/118842

### Non Patent Literature

[NPL 1] Yuya Nishida, et al., Nature 461,654-658
[NPL 2] Babak Razani et al., Cell Metabolism 2012, 15(4), 534-544
[NPL 3] Shaun Steele et al., PLOS Pathog 2013, vol. 9, Issue 8, e1003562
[NPL 4] Hye-Mi Lee et al., The Journal of Immunology (2011) 186(2), 1248-58

### SUMMARY

### Technical Problem

The present invention has been accomplished in light of the aforementioned problem of the prior art, and its object is to elucidate one aspect of the mechanism of ultraviolet light-induced inflammation and to provide a suppressing agent for ultraviolet light-induced inflammation.

### Solution to Problem

As a result of diligent research on the mechanism of skin inflammation, the present inventors have found for the first time that Atg5/Atg7-independent autophagy selectively contributes to reduced inflammation produced by ultraviolet rays, a cause of skin inflammation. Specifically, it was found that inducing autophagy in skin cultured cells could reduce skin inflammation produced by ultraviolet rays. With still further research on the relationship between ultraviolet light-induced inflammation and autophagy, the present invention was completed upon determining that skin inflammation produced by ultraviolet rays can be reduced by Atg5/Atg7-independent autophagy rather than by Atg5/Atg7-dependent autophagy which is the typical autophagy pathway. The present invention thus relates to the following.

[1] A suppressing agent of ultraviolet light-induced inflammation, comprising an alternative autophagy inducing agent as an active ingredient.

[2] The suppressing agent according to [1] above, wherein the ultraviolet light-induced inflammation is ultraviolet light-induced skin inflammation.

[3] The suppressing agent according to [2] above, wherein the suppressing agent is an external preparation for skin.

[4] The suppressing agent according to any one of [1] to [3] above, wherein the alternative autophagy inducing agent is at least one selected from the group consisting of Isodon japonicus extract, white nettle extract, wild oat extract, Chinese peony extract, camellia seed extract, Bulgarian rose water, sunflower oil, mangosteen extract, moringa extract and yukinoshita extract.

[5] The suppressing agent according to any one of [1] to [3] above, wherein the alternative autophagy inducing agent can selectively induce alternative autophagy.

[6] The suppressing agent according to [5] above, wherein the alternative autophagy inducing agent is Isodon japonicus extract.

[7] A screening method for suppressing agents of ultraviolet light-induced inflammation, using alternative autophagy activity as an indicator.

[8] The screening method according to [7] above, wherein the alternative autophagy activity is measured by the Rab9 gene expression level or protein level.

[9] The screening method according to [7] above, wherein autophagy activity is measured in a conventional type autophagy factor non-expressing cell line.

[10] The screening method according to [9] above, wherein the autophagy activity is measured based on the gene expression or protein level of one or more selected from the group consisting of Beclin1, Ulk1 and Rab9.

[11] The screening method according to [9] above, wherein the autophagy activity is measured by detection of autophagy vesicles.

[12] A method of evaluating resistance to ultraviolet damage, using alternative autophagy activity in skin as an indicator.

[13] The evaluating method according to [12] above, wherein alternative autophagy activity is measured by the gene expression or protein level of one or more selected from the group consisting of Beclin1, Ulk1 and Rab9.

[14] The evaluating method according to [13] above, wherein the alternative autophagy activity is measured by the Rab9 gene expression level or protein level.

[15] The evaluating method according to any one of [12] to [14] above, wherein the ultraviolet damage is ultraviolet light-induced skin inflammation.

[16] A method of suppressing or treating ultraviolet light-induced inflammation, comprising administering an effective dose of an alternative autophagy inducing agent to a subject in need of suppressing ultraviolet light-induced inflammation.

[17] The method according to [16] above, wherein the ultraviolet light-induced inflammation is ultraviolet light-induced skin inflammation.

[18] The method according to [17] above, wherein the alternative autophagy inducing agent is administered transdermally.

[19] The method according to any one of [16] to [18] above, wherein the alternative autophagy inducing agent is at least one selected from the group consisting of Isodon japonicus extract, white nettle extract, wild oat extract, Chinese peony extract, camellia seed extract, Bulgarian rose water, sunflower oil, mangosteen extract, moringa extract and yukinoshita extract.

[20] The method according to any one of [16] to [18] above, wherein the alternative autophagy inducing agent can selectively induce alternative autophagy.

[21] The method according to [20] above, wherein the alternative autophagy inducing agent is Isodon japonicus extract.

[22] An alternative autophagy inducing agent for use in suppressing or treating ultraviolet light-induced inflammation.

[23] The alternative autophagy inducing agent according to [22] above, wherein the ultraviolet light-induced inflammation is ultraviolet light-induced skin inflammation.

[24] The alternative autophagy inducing agent according to [23] above, to be used for topical skin use.

[25] The alternative autophagy inducing agent according to any one of [22] to [24] above, wherein the alternative autophagy inducing agent is at least one selected from the group consisting of Isodon japonicus extract, white nettle extract, wild oat extract, Chinese peony extract, camellia seed extract, Bulgarian rose water, sunflower oil, mangosteen extract, moringa extract and yukinoshita extract.

[26] The alternative autophagy inducing agent according to any one of [22] to [24] above, wherein the alternative autophagy inducing agent can selectively induce alternative autophagy.

[27] One or more extracts selected from the group consisting of Isodon japonicus extract, white nettle extract, wild oat extract, Chinese peony extract, camellia seed extract, Bulgarian rose water, sunflower oil, mangosteen extract, moringa extract and yukinoshita extract, for use in suppressing or treating ultraviolet light-induced inflammation through inducing alternative autophagy.

[28] Isodon japonicus extract, for use in suppressing or treating ultraviolet light-induced inflammation through selectively inducing alternative autophagy.

[29] Use of an alternative autophagy inducing agent for production of a therapeutic or suppressing agent for ultraviolet light-induced inflammation.

[30] The use according to [29] above, wherein the ultraviolet light-induced inflammation is ultraviolet light-induced skin inflammation.

[31] The use according to [30] above, wherein the suppressing agent is an external preparation for skin.

[32] The use according to any one of [29] to [31] above, wherein the alternative autophagy inducing agent includes at least one selected from the group consisting of Isodon japonicus extract, white nettle extract, wild oat extract, Chinese peony extract, camellia seed extract, Bulgarian rose water, sunflower oil, mangosteen extract, moringa extract and yukinoshita extract.

[33] The use according to any one of [29] to [31] above, wherein the alternative autophagy inducing agent can selectively induce alternative autophagy.

[34] The use according to [33] above, wherein the alternative autophagy inducing agent is Isodon japonicus extract.

### Advantageous Effects of Invention

Ultraviolet light-induced inflammation can be suppressed by inducing autophagy, and particularly alternative autophagy. Ultraviolet light-induced inflammation is one of the causes of acute and chronic damage, and such ultraviolet damage can be reduced by reducing ultraviolet light-induced inflammation. Since alternative autophagy contributes to reduced ultraviolet light-induced inflammation, using alternative autophagy activity as an indicator allows screening of suppressing agents of ultraviolet light-induced inflammation and evaluation of resistance to ultraviolet light-induced inflammation.

### BRIEF DESCRIPTION OF DRAWINGS

FIG. 1A is a graph showing increase in IL-1β production induced by ultraviolet ray by addition of the autophagy inhibitor 3-MA. FIG. 1B is a graph showing suppression of IL-1β production induced by ultraviolet ray by addition of the autophagy inducing agent rapamycin.

FIG. 2A is a graph showing suppressed Atg5 expression by introduction of Atg5-targeted siRNA into cells. FIG. 2B is a graph showing suppressed Atg7 expression by introduction of Atg7-targeted siRNA into cells. FIG. 2C is a graph showing suppressed Beclin1 expression by introduction of Beclin1-targeted siRNA into cells.

FIG. 3A is a graph showing that IL-1β production induced by ultraviolet ray was unchanged in cells in which Atg5-targeted siRNA was introduced. FIG. 3B is a graph showing that IL-1β production induced by ultraviolet ray was unchanged in cells in which Atg7-targeted siRNA was introduced. FIG. 3C is a graph showing that IL-1β production induced by ultraviolet ray was significantly increased in cells in which Beclin1-targeted siRNA was introduced.

### DESCRIPTION OF EMBODIMENTS

The ultraviolet light-induced inflammation suppressing agent of the invention comprises an alternative autophagy inducing agent. Inflammation to be suppressed according to the invention is inflammation elicited by ultraviolet rays, and more preferably ultraviolet light-induced skin inflammation.

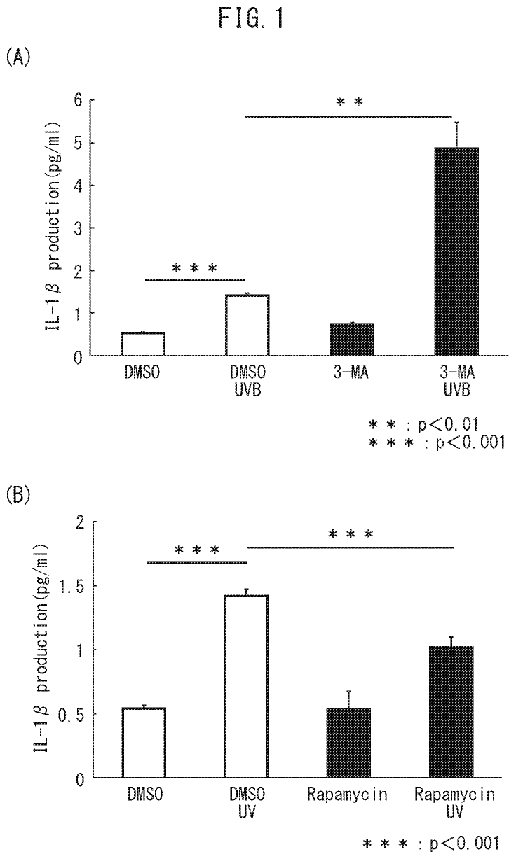

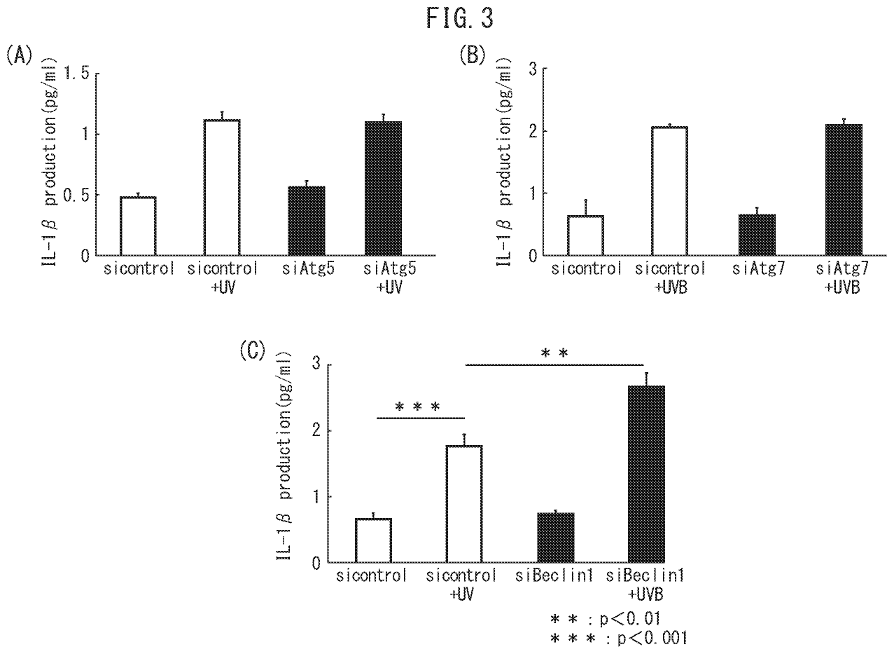

Inflammation is a syndrome characterized by redness, heat, swelling and pain, and it is caused by external invasion such as microbe infection, contaminant infiltration, heavy metal exposure and ultraviolet irradiation, as well as by internal stimulants released by necrotic cells and the like. The syndrome of inflammation takes place in all kinds of biological tissues, with different causes and inflammatory mechanisms depending on the tissue. Skin tissue, in particular, is exposed to the exterior of a living body and acts as a barrier forming a boundary between the external world and the organism. Lowering of the skin barrier function invites transdermal sensitization by a variety of external invasion factors, causing skin inflammation such as dermatitis and allergic disease. It is known that filaggrin expressed by keratinocytes plays an important role in formation of the skin barrier. It has also been reported that mutations in filaggrin are associated with onset of atopic dermatitis, a type of inflammatory disease. Skin inflammation therefore has characteristic mechanisms which differ from inflammation in internal body tissues. The present inventors have found that the alternative autophagy pathway of the invention acts in a suppressive manner against inflammation and especially ultraviolet light-induced inflammation (Examples, FIGS. 1 and 3). The present inventors have also found that the Atg5-dependent autophagy pathway which has conventionally been considered to contribute to tissue inflammation is not involved in ultraviolet light-induced inflammation (Examples, FIG. 3). Since NPL 4 has shown Atg5-dependent autophagy to have a suppressive effect against skin inflammation induced by MALP-2, it is not possible to predict that the Atg5-dependent autophagy pathway is not involved in ultraviolet light-induced inflammation. It is well known that although skin inflammation and ultraviolet light-induced inflammation both eventually lead to inflammatory cytokine production, the generating mechanisms are different. Experiments conducted by the present inventors were the first to suggest that skin inflammation is different from ultraviolet light-induced inflammation in the regulating mechanisms.

According to the invention, the term “ultraviolet rays” may refer to UV-A, UV-B or UV-C but it preferably refers to UV-A and UV-B in particular because they reach the ground and cause inflammation of the skin. While both UV-A and UV-B are known to cause inflammation, UV-B may be referred to since it is a greater contributor to inflammation.

The symptoms of inflammation caused by ultraviolet rays include erythema and blistering, as well as eczema in severe cases. Eczema in the acute stage produces morphological changes such as erythema, papules, vesicles, pustules, sores, scabs and exfoliation, and although it eventually heals, if acute eczema is left to become chronic without treatment it can result in lichenization and pigmentation. Skin diseases caused by inflammation as a result of ultraviolet rays include solar dermatitis, solar cheilitis, photocontact dermatitis, chronic photosensitive dermatitis, Berloque dermatitis, photoallergy, photosensitive drug eruption, solar urticaria, xeroderma pigmentosum, dermatomyositis, porphyria, pellagra, chronic photodermatosis, polymorphous light eruption and erythematosus. Ultraviolet rays also cause eye inflammation, producing ultraviolet keratitis which is inflammation that can lead to cataracts. Therefore, the ultraviolet light-induced inflammation suppressing agent or alternative autophagy inducing agent of the invention can treat, reduce, suppress and prevent the aforementioned skin diseases and eye disease caused by inflammation, and can be considered as treatment agents, alleviating agents, suppressing agents and prophylactic agents for the skin and eye diseases. Inflammation in the skin also accelerates secretion of melanocyte-stimulating hormone which is associated with browning and blemish formation in the skin, while chronic inflammation is also known to promote senescence. Ultraviolet light-induced inflammation suppressing agents or alternative autophagy inducing agents of the invention can therefore be considered to be sunburn suppressing agents, skin whiteners and skin aging preventers.

A subject who uses an ultraviolet light-induced inflammation suppressing agent of the invention may be any subject who is in need of alleviation of ultraviolet light-induced inflammation. An ultraviolet light-induced inflammation suppressing agent of the invention can be administered to persons as well as athletes and workers that are active outdoors, persons that need to avoid sunburn for cosmetic or health reasons, and patients suffering from diseases with ultraviolet damage. An alternative autophagy inducing agent can be administered to a subject with reduced alternative autophagy activity.

An ultraviolet light-induced inflammation suppressing agent of the invention can suppress production of one or more inflammatory cytokines selected from among IL-1β, IL-1α, TNF-α, IL-4, IL-6, IL-8, IL-12, IL-18, TSLP and GM-CSF, or chemokines such as CCL2 or CXCL10, or inflammatory mediators such as PGE₂, whose secretion is increased in keratinocytes due to ultraviolet irradiation. The ultraviolet light-induced inflammation suppressing agent or alternative autophagy inducing agent can therefore be considered to be an inflammatory cytokine suppressing agent or inflammatory mediator suppressing agent.

Alternative autophagy is an intracellular cleaning mechanism in which autophagosomes are formed and fused with lysosomes and intracellular components taken up into the autophagosomes are decomposed, without using the autophagy-associated molecule Atg5. Alternative autophagy can therefore be considered to be Atg5- and/or Atg7-independent autophagy. While it is not our intention to be restricted to any particular theory, it is though that a cause of inflammation is eliminated by decomposing denatured abnormal proteins and induced inflammatory substances resulting from the effect of ultraviolet rays through the action of alternative autophagy. Throughout the present specification, the conventional type of autophagy, i.e. Atg5- and/or Atg7-dependent autophagy, will be referred to as “conventional autophagy” so as to distinguish it from alternative autophagy.

An alternative autophagy inducing agent may be any substance that can accelerate activation of alternative autophagy. While substances that can selectively accelerate alternative autophagy are preferred, they may also be substances that non-selectively accelerate another autophagy type as well. Alternative autophagy inducing agents may therefore include conventional autophagy inducing agents, or from a different aspect they may exclude conventional autophagy inducing agents. Inducing agents that are able to induce both autophagy other than alternative autophagy (for example, conventional autophagy) and alternative autophagy may be referred to as alternative autophagy non-selective inducing agents, while inducing agents that induce primarily alternative autophagy may be referred to as alternative autophagy selective inducing agents. Alternative autophagy selective inducing agents include the benzothiophene compounds listed in PTL 1, and Francisella tularensis mentioned in NPL 3. The screening method of the invention has demonstrated that Isodon japonicus extract is also a substance that acts as an alternative autophagy selective inducing agent. Non-selective inducing agents of alternative autophagy include rapamycin, verapamil and clonidine. The screening method of the invention has demonstrated that Isodon japonicus extract, white nettle extract, wild oat extract, Chinese peony extract, camellia seed extract, Bulgarian rose water, sunflower oil, mangosteen extract, moringa extract and yukinoshita extract are substances that act as alternative autophagy non-selective inducing agents. However, it is not our intention to restrict autophagy inducing agents to these specific compounds or microbes or extracts.

An alternative autophagy inducing agent of the invention, or an ultraviolet light-induced inflammation suppressing agent containing the inducing agent, can be added as an active ingredient to a functional food, cosmetic or medicament for the purpose of alleviating ultraviolet light-induced inflammation. Such a cosmetic to which an alternative autophagy inducing agent is formulated may be a sunscreen, cosmetic water, essence, beauty cream, aftercare lotion or sun oil, and any cosmetic that is to be applied to skin. medicament include anti-inflammatory external preparations for skin, and anti-inflammatory oral drugs. Since alternative autophagy inducing agents were found to be effective for ultraviolet light-induced inflammation, they are preferably formulated as external preparations for skin that can be directly applied onto the skin. An alternative autophagy inducing agent may also be formulated to an eye lotion for prevention of cataract, for use in alleviating and suppressing ultraviolet damage. An alternative autophagy inducing agent or a skin inflammation suppressing agent containing the inducing agent may also be formulated with arbitrary compounding ingredients used in cosmetics and medicament as necessary so long as its effect is not impaired. Examples of arbitrary compounding ingredients include oils, surfactants, powders, coloring materials, water, alcohols, thickeners, chelating agents, silicones, antioxidants, ultraviolet absorbers, humectants, perfumes, drug components, antiseptic agents, pH adjustors and neutralizing agents. Anti-inflammatory components and whitening components may also be included, as examples of additional drug components.

The present invention also relates to a screening method for ultraviolet light-induced inflammation suppressing agents using alternative autophagy activity as an indicator. The screening method comprises a step of adding a candidate drug to cultured cells, and a step of measuring the alternative autophagy activity in the cultured cells. When alternative autophagy activity has increased relative to a control, the candidate drug may be selected as an ultraviolet light-induced inflammation suppressing agent or an alternative autophagy inducing agent. The candidate drug used may be any desired substance, examples of which include substances listed in drug candidate compound libraries or cosmetic material libraries, and they include not only compounds but also mixtures and extracts.

The cultured cells used may be any desired cells, from an established cell line or as primary cultured cells or subcultured cells isolated and cultured from tissue. From the viewpoint of evaluating the effects of ultraviolet rays, body cells that have been exposed to ultraviolet rays, such as skin cells or eye cells, may be used. Examples of skin cells include keratinocytes, pigment cells and dermal fibroblasts, and examples of eye cells include corneal epithelial cells and retinal epithelial cells. A three-dimensional cultured skin model with cultured layers of skin culture cells may also be used. As a different embodiment, the cultured cells used in the screening method may be a conventional autophagy factor non-expressing line that lacks expression of at least one conventional autophagy factor. Such a cell line may be a gene-knockout cell line created by genome editing, such as point mutation, homologous recombination or a CRISPR-Cas9 system, or it may be a knockdown cell line with inhibited gene expression by introduction of siRNA. Using a conventional autophagy factor non-expressing cell line allows screening of active inducing agents of alternative autophagy even when using a marker that is not specific for alternative autophagy but also detects conventional autophagy. As examples, Beclin1 or Ulk1 contribute to both alternative autophagy and conventional autophagy, and therefore using their gene expression level or protein level in a conventional autophagy factor non-expressing cell line as the autophagy activity indicator allows screening of alternative autophagy-inducing drugs. Examples of conventional autophagy factor non-expressing lines include Atg5 and/or Atg7 gene knockout cell lines, and Atg5 and/or Atg7 gene knockdown cell lines. Instead of using gene expression level or protein level as an indicator, it is still possible to use intracellular autophagy vesicles as an indicator of autophagy activity. Autophagy vesicles are also known as autophagosomes. Autophagosomes can be observed under a microscope, for example, by using LC, etc., as a marker.

The screening method of the invention has allowed selection of the following plant extracts: Isodon japonicus extract, white nettle extract, wild oat extract, Chinese peony extract, camellia seed extract, Bulgarian rose water, sunflower oil, mangosteen extract, moringa extract and yukinoshita extract, as ultraviolet light-induced inflammation suppressing agents or alternative autophagy inducing agents, from among a cosmetic material library. Another aspect of the invention, therefore, relates to an ultraviolet light-induced inflammation suppressing agent or alternative autophagy inducing agent that comprises one or more plant extracts selected from the group consisting of Isodon japonicus extract, white nettle extract, wild oat extract, Chinese peony extract, camellia seed extract, Bulgarian rose water, sunflower oil, mangosteen extract, moringa extract and yukinoshita extract. These extracts may have conventional autophagy-inducing activity as well. However, Isodon japonicus extract does not have conventional autophagy-inducing activity while exhibiting powerful alternative autophagy-inducing activity, and it can therefore be considered to be an alternative autophagy selective inducing agent.

The plant body or extract from a plant to be used for the invention is any part of the plant body (flower, flowering spike, peel, fruit, stem, leaf, stalk, side leaf, trunk, bark, rhizome, root bark, root, seed or the whole plant) used either directly or dried and pulverized into a dry powder, or extracted with a solvent from the plant part either directly or after drying and pulverizing. The extraction site may be the leaves, roots, stem or flowers, with no particular limitation to these sites.

For an extract, the extraction solvent used for extraction may be any solvent commonly used for extraction, and particularly there may be used alcohols such as methanol, ethanol or 1,3-butylene glycol, or organic solvents such as water-containing alcohols, acetone or ethyl acetate, either alone or in combinations, among which alcohols and water-containing alcohols are especially preferred, with methanol, ethanol, 1,3-butylene glycol, water-containing ethanol and water-containing 1,3-butylene glycol being most preferred. The solvent is preferably used at room temperature or a temperature below the boiling point of the solvent. Water-containing 1,3-butylene glycol contains 1,3-butylene glycol at 20 to 80 mass %, preferably 30 to 70 mass % and even more preferably 40 to 60 mass %. An example for use as an extraction solvent is a 50 mass % 1,3-butylene glycol aqueous solution.

The extraction process is not particularly limited, but it will usually be carried out in a range from ordinary temperature to the boiling point of the solvent under ordinary pressure, and following extraction, either filtration or an ion exchange resin may be used for adsorption, decoloration and purification into a solution, paste, gel or powder form. In most cases this form may be utilized directly, but if necessary, purifying treatment such as deodorization or decoloration may be performed, within ranges that do not alter the effect, and the means for purifying treatment such as deodorization or decoloration may be an active carbon column or the like, with commonly employed means being selected as appropriate and desired depending on the extracted substance. The extracts to be used for the invention are all commercially available as cosmetic materials, and their production methods may differ depending on the distributor.

Isodon japonicus is a plant of the family Lamiaceae, genus Isodon which is native to Japan, being indigenous to Honshu, Shikoku and Kyushu islands. Isodon japonicus extract is an extract obtained using the aforementioned solvents for extraction from the entire plant of Isodon japonicus. As an example, Isodon japonicus extract can be obtained by extraction from the dried Isodon japonicus plant, which is commercially available as a galenical, using water, propylene glycol, 1,3-butylene glycol or a mixture thereof. The Japanese name for Isodon japonicus means “life-extending grass” and has long been used as a folk medicine in Japan. It is known to have moisturizing, blood circulation promoting, astringent and antibacterial drug effects, and is also used as a bitter stomach medicine.

White nettle (scientific name: Lamium album Linne) is a plant of the family Lamiaceae that is native to Japan, being indigenous to a wide region including Hokkaido, Honshu, Shikoku, Kyushu, the Korean peninsula and China. White nettle extract is an extract obtained by extraction from the flowers, stems or leaves of white nettle using the extraction solvents mentioned above. As an example, it may be obtained from the flowers, stems or leaves of white nettle by extraction with water, propylene glycol, 1,3-butylene glycol or a mixture thereof.

Wild oat is a plant of the family Poaceae, genus Avena, being native in the zone from Europe to western Asia, with numerous wild varieties and cultivated varieties extant across wide regions. Wild varieties of wild oat (scientific name: Avena fatua) are common, and the cultivated variety of oats (scientific name: Avena sativa) can also be used as a starting material for extracts. Wild oat extract is an extract obtained by extraction from the stems, leaves, seeds or grains using the extraction solvents mentioned above. As an example, it may be obtained from wild oat grains by extraction with water, propylene glycol, 1,3-butylene glycol or a mixture thereof.

Chinese peony is a perennial herb of the family Paeoniaceae, native to the northern regions of the Asian continent. Species used for Chinese peony extract include Chinese peony (Paeonia lactijlora Pallas (Paeonia albijlora Pallas var. trichocarpa Bunge)) and its related plants (Paeoniaceae). The extract is obtained from the plant body of Chinese peony by extraction using the extraction solvents mentioned above. As an example, it may be obtained from Chinese peony roots by extraction with water, propylene glycol, 1,3-butylene glycol or a mixture thereof.

Camellia (scientific name: Camellia japonica) is an evergreen tree of the family Theaceae, genus Camellia, which is native to Japan. It is indigenous to Honshu, Shikoku, Kyushu and the southwestern Japanese islands, as well as to the southern Korean peninsula and Taiwan. Camellia seed extract is obtained by extraction from camellia seeds using the extraction solvents mentioned above. As an example, it may be obtained by extraction from the powder or dry powder of camellia seeds using water, propylene glycol, 1,3-butylene glycol or a mixture thereof.

Rose is a general term for plants of the Family Rosaceae, genus Rosa, which are indigenous to a wide range in the northern hemisphere temporal zone. A large variety of species of rose exist, and rose water is extracted from rose leaves of different species by steam distillation. The Damascus rose variety (Rosa damascena), in particular, is suitable as a starting material for rose water because of its excellent aroma. Rose water obtained from Bulgarian Damascus rose is known as Bulgarian rose water and is marketed as a cosmetic material.

FLORASUN 90 is a type of sunflower oil marketed as a cosmetic material. Sunflower (scientific name: Helianthus annuus) is an annual grass of the family Compositae, native to North America. Sunflower seeds are rich in fats and oils and the sunflower oil can be obtained by oil pressing. Sunflower oil differs depending on the types of unsaturated fatty acids in the variety, and sunflower oil having high oleic acid content is especially preferred.

Mangosteen (scientific name: Garcinia mangostana) is a plant of the Family Clusiaceae, genus Garcinia which is native to Southeast Asia. Mangosteen extract is obtained by extraction from the capsules, peel, fruit, stems, leaves, branches, side leaves, trunk, bark, rhizomes, root bark, roots, seeds or entire plant of mangosteen, using the extraction solvents mentioned above. As an example, it may be obtained from mangosteen peel by extraction with water, propylene glycol, 1,3-butylene glycol or a mixture thereof.

Moringa is a plant of the genus Moringa, which is indigenous across Africa and throughout the tropical and subtropical regions of South Asia. The drumstick tree (scientific name: Moringa oleifera Lam) is a particularly widely cultivated species. Moringa extract is an extract obtained by extraction from the leaves, flowers, bark, fruit, seeds or roots using the extraction solvents mentioned above. As an example, it may be obtained from drumstick tree leaves or roots by extraction with water, propylene glycol, 1,3-butylene glycol or a mixture thereof.

Yukinoshita (Saxifraga stolonifera) is a plant of the genus Saxifraga which is a perennial herb indigenous to Japan and China. Yukinoshita extract is an extract obtained by extraction from the entire plant, leaves, stems, roots, flowers or seeds using the extraction solvents mentioned above. As an example, it may be obtained from yukinoshita leaves by extraction with water, propylene glycol, 1,3-butylene glycol or a mixture thereof.

Another aspect of the invention relates to a method for evaluating resistance to skin inflammation using alternative autophagy activity as an indicator. According to this method it is possible to evaluate resistance to skin inflammation by measuring alternative autophagy activity in a skin sample from a test subject. Cosmetics can be selected depending on the evaluated resistance to skin inflammation. For example, a subject evaluated to have low alternative autophagy activity may be advised to use a cosmetic with low irritation that is unlikely to produce skin inflammation. Based on resistance to ultraviolet light-induced inflammation as one type of skin inflammation, a more powerful sunscreen or a cosmetic such as aftercare lotion may be suggested for a subject evaluated to have low alternative autophagy activity, or a functional food, cosmetic or drug containing an alternative autophagy inducing agent may be suggested.

Alternative autophagy activity can be determined using expression or activity of a factor involved in alternative autophagy as an indicator. Factors involved in alternative autophagy include Beclin1, Ulk1 and Rab9, and for example, it is possible to detect the changes in the gene and protein expression of these factors Beclin1, Ulk1 and Rab9 and to visualize them by methods such as immunostaining. From the viewpoint of specifically measuring alternative autophagy, it is preferred to use Rab9 which is thought not to be involved in conventional autophagy. According to a different aspect, alternative autophagy activity can be detected using organelles and substances involved in alternative autophagy, such as lysosomes, autophagosomes or proteins deriving from lysosomes or autophagosomes. Aggregation of lysosome-derived proteins LAMP1 or LAMP2 in Atg5- and/or Atg7-knockdown or knockout cells can also be visualized by methods such as immunostaining.

### Examples

### Effects of Autophagy Inhibitors and Inducing Agents on Ultraviolet-Inducible Inflammation

Normal human epidermal keratinocytes (NHEK) (Kurabo Industries, Ltd.) were seeded in a 6-well plate and cultured to subconfluence using epidermal keratinocyte growth medium (EpiLife-KG2; Gibco Co). The medium was then exchanged with medium containing 3-methyladenine (3-MA) (by R&D Co., final concentration: 1 mM), as a known autophagy inhibitor and medium containing rapamycin (by Enzo Life Science, final concentration: 0.5 μM) as a known autophagy inducing agent, and culturing was continued for 3 hours. After culturing, the medium was discarded, PBS was added and irradiation was carried out with ultraviolet rays (290 to 315 nm) at a radiation intensity of 20 mJ/cm². Following ultraviolet irradiation, culturing was continued under the medium conditions described above including 3-MA or rapamycin, and after 48 hours the culture supernatant of each was obtained. The IL-1β concentration of the obtained culture supernatant was evaluated using a Quantikine Human IL-1β ELISA Kit (R&D co.) (FIGS. 1A and B). Statistical significance testing was conducted using Student's or Welch's t test.

Since the IL-1β concentration increased under ultraviolet irradiation, this indicated that inflammation had been induced. When the autophagy inhibitor 3-methyladenine was added, the IL-1β concentration after ultraviolet irradiation increased approximately 3-fold. However, addition of rapamycin which is known as an autophagy inducing agent significantly decreased the IL-1β after ultraviolet irradiation. These results indicated that inducing autophagy can alleviate ultraviolet ray-inducible inflammation.

### Identifying Type of Autophagy Contributing to Alleviation of Ultraviolet-Inducible Inflammation

Small interfering RNA (siRNA) for Atg5, Atg7 and Beclin1 were purchased from Invitrogen Co. Their sequences are as shown in Table 1.

TABLE 1

siAtg5 nucleotide sequence (5′ → 3′)
GGUUUGGACGAAUUCCAACUUGUUU (SEQ ID NO: 1)
AAACAAGUUGGAAUUCGUCCAAACC (SEQ ID NO: 2)
siAtg7 nucleotide sequence (5′ → 3′)
GCCGUCAUUGCUGCAAGCAAGAGAA (SEQ ID NO: 3)
UUCUCUUGCUUGCAGCAAUGACGGC (SEQ ID NO: 4)
siBeclin1 nucleotide sequence (5′ → 3′)
CCAAUAAGAUGGGUCUGAAAUUUCA (SEQ ID NO: 5)
UGAAAUUUCAGACCCAUCUUAUUGG (SEQ ID NO: 6)

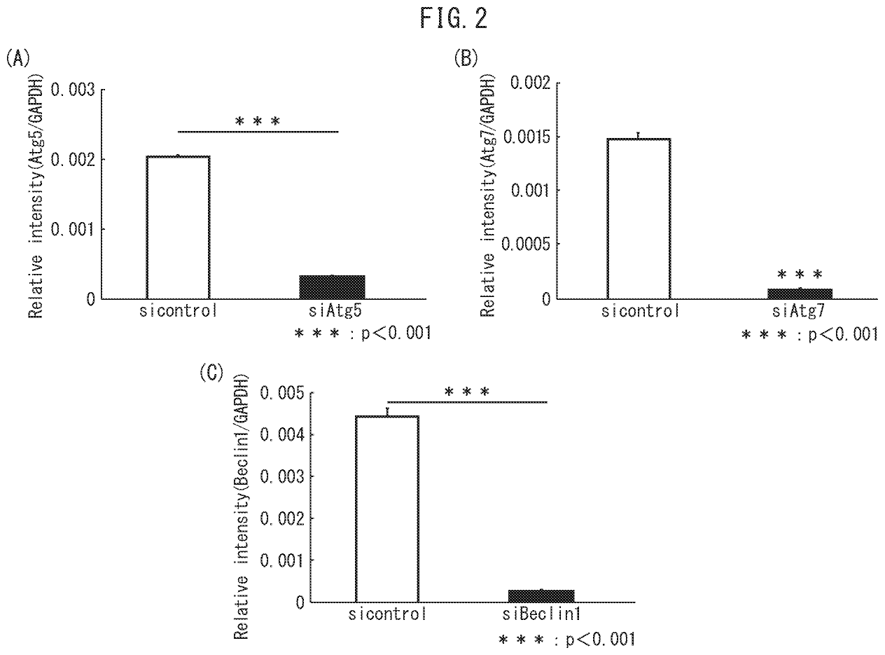

An Amaxa Human Keratinocyte Nucleofector Kit (Lonza) was used to transfect NHEK cells (8.0×10⁵ cells) with siRNA to a final concentration of 200 nM. The knockdown efficiency was confirmed by the mRNA expression level of Atg5, Atg7 and Beclin1 using an RT-PCR method (FIGS. 2A, B and C). The RT-PCR primers used were primers by Sigma-Aldrich, and GAPDH (Sigma-Aldrich) was used as an internal standard for standardization of the expression levels. The primer sequences are shown in Table 2.

TABLE 2

| Primer | Sequence |
|---|---|
| Atg5 Forward | caacttgtttcacgctatatcagg |
| | (SEQ ID NO: 7) |
| Atg5 Reverse | cactttgtcagttaccaacgtca |
| | (SEQ ID NO: 8) |
| Atg7 Forward | ccgtggaattgatggtatctg |
| | (SEQ ID NO: 9) |
| Atg7 Reverse | tcatccgatcgtcactgct |
| | (SEQ ID NO: 10) |
| Beclin-1 Forward | ggatggtgtctctcgcagat |
| | (SEQ ID NO: 11) |
| Beclin-1 Reverse | ttggcactttctgtggacat |
| | (SEQ ID NO: 12) |
| GAPDH Forward | agccacatcgctcagacac |
| | (SEQ ID NO: 13) |
| GAPDH Reverse | gcccaatacgaccaaatcc |
| | (SEQ ID NO: 14) |

The siRNA-treated NHEK cells were seeded into a 6-well plate and cultured for 24 hours. The medium was then discarded, PBS was exchanged, and irradiation was carried out with ultraviolet rays (290 to 315 nm) at a radiation intensity of 15 mJ/cm². Following ultraviolet irradiation, the medium was again exchanged and culturing was continued for 48 hours to obtain a culture supernatant. The IL-1β concentration of the obtained culture supernatant was evaluated using a Quantikine Human IL-1β ELISA Kit (R&D co.) (FIGS. 3A, B and C). Student's t-test was used as the statistical significance test.

Using Atg5-, Atg7- and Beclin1-targeted siRNA, the gene expressions of all were shown to be suppressed (FIGS. 2A, B and C). Atg5 and Atg7 are believed to be proteins essential for Atg5/Atg7-dependent autophagy, while Beclin1 is believed to be a protein essential for autophagy including both the Atg5/Atg7-dependent autophagy and alternative autophagy pathways. Presumably, therefore, in cells with suppressed Atg5 and Atg7 gene expression only the Atg5/Atg7-dependent autophagy fails to function, whereas in cells with suppressed Beclin1 gene expression, the autophagy pathway itself, including Atg5/Atg7-dependent autophagy and alternative autophagy, fails to function.

In keratinocytes with suppressed Atg5 and Atg7 expression, there was no change in IL-1β concentration after irradiation of ultraviolet rays (FIGS. 3A and 3B). In keratinocytes with suppressed Beclin1 expression, on the other hand, the IL-1β concentration after ultraviolet irradiation was significantly increased (FIG. 3C). It was thus suggested that Atg5/Atg7-dependent autophagy is not at all involved in alleviation of inflammation caused by ultraviolet rays, whereas alternative autophagy does contribute to alleviation of inflammation caused by ultraviolet rays.

### Screening Method for Alternative Autophagy Inducing Agents

Normal 293T cells and Atg5-deficient 293T cells were seeded into DMEM+10% FBS medium at 1×10⁴ cells/well and cultured for 2 days. After culturing, the medium was exchanged with medium containing the test substance, and an autophagy monitor pigment (Dojindo) was added to 1 μM concentration and the autophagy activity was examined. A total of 20 test substances among 212 different test substances induced autophagy activity to the same degree in both the normal 293T cells and the Atg5-deficient 293T cells.

The selected 20 test substances were then measured for autophagy activity in normal human epidermal keratinocytes (Hacat). After treatment with the aforementioned Atg5 knockdown siRNA, the Atg5 knockdown Hacat cells were obtained. Normal Hacat cells and the Atg5 knockdown Hacat cells were each seeded in DMEM+10% FBS medium at 1×10⁴ cells/well and cultured for 2 days. After culturing, the medium was exchanged with medium containing the test substance, and an autophagy monitor pigment (Dojindo) was added to 1 μM concentration and the autophagy activity was examined. A total of 10 test substances among 20 different test substances induced autophagy activity to the same degree in both the normal Hacat cells and the Atg5-knockdown Hacat cells. It was thus suggested that the 10 test substances induce Atg-independent autophagy in epidermal keratinocytes.

The selected 10 test substances were measured for ability to induce conventional autophagy activity and alternative autophagy (Atg5/Atg7-independent autophagy) activity. Specifically, normal Hacat cells were cultured in medium with the test substance added, and anti-LC3-II antibody (Cosmo bio) was used for immunostaining. These were observed under a fluorescent microscope, and the change in fluorescent brightness over the entire visual field compared to the non-test substance-added control group was recorded. Since LC3-II is a marker for conventional autophagy, ability to induce conventional autophagy activity was demonstrated when fluorescent brightness increased compared to the non-test substance-added control group. Atg5 knockdown Hacat cells were then cultured in medium with addition of the test substance, and anti-Lamp1 antibody (Abcam) was used for immunostaining. These were observed under a fluorescent microscope, and the change in fluorescent brightness over the entire visual field compared to the non-test substance-added control group was recorded. Since Lamp1 is a marker for autophagy, ability to induce Atg-independent autophagy activity was demonstrated when fluorescent brightness increased compared to the non-test substance-added control group. The results are shown in the following table.

TABLE 3

| | Conventional | Alternative |
|---|---|---|
| | autophagy- | autophagy- |
| | inducing activity | inducing activity |
| 29 | − | ++ |
| 45 | + | ++ |
| 62 | ++ | + |
| 95 | + | + |
| 129 | + | ++ |
| 156 | + | ++ |
| 157 | + | + |
| 174 | + | + |
| 179 | + | + |
| 183 | + | + |

29: Isodon Japonicus extract, 45: white nettle extract, 62: wild oat extract, 95: Chinese peony extract BG, 129: camellia seed extract BG, 156: Bulgarian rose water, 157: FLORASUN 90,174: mangosteen extract BG, 179: moringa extract G, 183: yukinoshita extract BG

Powerful alternative autophagy-inducing activity was seen with test substance Nos. 29, 34, 129 and 156. Test substance 29 did not induce conventional autophagy but was able to induce only alternative autophagy activity.

### SEQUENCE LISTING

### C:\Users\SYJF05\Documents\P190362WO_SSD.txt

1. A method for suppressing ultraviolet light-induced inflammation, comprising applying an alternative autophagy inducing agent as an active ingredient to skin.

2. The method according to claim 1, wherein the ultraviolet light-induced inflammation is ultraviolet light-induced skin inflammation.

3. (canceled)

4. The method according to claim 1, wherein the alternative autophagy inducing agent is at least one selected from the group consisting of Isodon japonicus extract, white nettle extract, wild oat extract, Chinese peony extract, camellia seed extract, Bulgarian rose water, sunflower oil, mangosteen extract, moringa extract and yukinoshita extract.

5. The method according to claim 1, wherein the alternative autophagy inducing agent can selectively induce alternative autophagy.

6. The method according to claim 5, wherein the alternative autophagy inducing agent is Isodon japonicus extract.

7. A screening method for suppressing agents of ultraviolet light-induced inflammation, using alternative autophagy activity as an indicator.

8. The screening method according to claim 7, wherein the alternative autophagy activity is measured by the Rab9 gene expression level or protein level.

9. The screening method according to claim 7, wherein alternative autophagy activity is measured in in a conventional autophagy factor non-expressing cell line.

10. The screening method according to claim 9, wherein the autophagy activity is measured based on the gene expression or protein level of one or more selected from the group consisting of Beclin1, Ulk1 and Rab9.

11. The screening method according to claim 9, wherein the autophagy activity is measured by detection of autophagy vesicles.

12. A method of evaluating resistance to ultraviolet damage, comprising measuring alternative autophagy activity in skin, and using the alternative autophagy activity as an indicator.

13. The evaluating method according to claim 12, wherein alternative autophagy activity is measured by the gene expression or protein level of one or more selected from the group consisting of Beclin1, Ulk1 and Rab9.

14. The evaluating method according to claim 13, wherein the alternative autophagy activity is measured by the Rab9 gene expression level or protein level.

15. The evaluating method according to claim 12, wherein the ultraviolet damage is ultraviolet light-induced skin inflammation.


SEQUENCE LISTING (14 sequences)

SEQ ID NO: 1 - RNA, 25 nt, Artificial Sequence
  siRNA
gguuuggacgaauuccaacuuguuu

SEQ ID NO: 2 - RNA, 25 nt, Artificial Sequence
  siRNA
aaacaaguuggaauucguccaaacc

SEQ ID NO: 3 - RNA, 25 nt, Artificial Sequence
  siRNA
gccgucauugcugcaagcaagagaa

SEQ ID NO: 4 - RNA, 25 nt, Artificial Sequence
  siRNA
uucucuugcuugcagcaaugacggc

SEQ ID NO: 5 - RNA, 25 nt, Artificial Sequence
  siRNA
ccaauaagaugggucugaaauuuca

SEQ ID NO: 6 - RNA, 25 nt, Artificial Sequence
  siRNA
ugaaauuucagacccaucuuauugg

SEQ ID NO: 7 - DNA, 24 bp, Artificial Sequence
  Primer
caacttgtttcacgctatatcagg

SEQ ID NO: 8 - DNA, 23 bp, Artificial Sequence
  Primer
cactttgtcagttaccaacgtca

SEQ ID NO: 9 - DNA, 21 bp, Artificial Sequence
  Primer
ccgtggaattgatggtatctg

SEQ ID NO: 10 - DNA, 19 bp, Artificial Sequence
  Primer
tcatccgatcgtcactgct

SEQ ID NO: 11 - DNA, 20 bp, Artificial Sequence
  Primer
ggatggtgtctctcgcagat

SEQ ID NO: 12 - DNA, 20 bp, Artificial Sequence
  Primer
ttggcactttctgtggacat

SEQ ID NO: 13 - DNA, 19 bp, Artificial Sequence
  Primer
agccacatcgctcagacac

SEQ ID NO: 14 - DNA, 19 bp, Artificial Sequence
  Primer
gcccaatacgaccaaatcc